(19) United States
(12) Patent Application Publication
(10) Pub. No.: US 2009/0010916 A1
(43) Pub. Date: Jan. 8, 2009

(54) C-1 Inhibitor prevents non-specific plasminogen activation by a prourokinase mutant without impeding fibrin-specific fibrinolysis

(76) Inventors: Victor Gurewich, Cambridge, MA (US); Ralph Pannell, Brighton, MA (US)

Correspondence Address: Kevin M. Farrell;Pierce Atwood, Suite 350, One New Hampshire Avenue, Portsmouth, NH, 03801, US

(21) Appl. No.: 12/215,966

(22) Filed: Jul. 1, 2008

Related U.S. Application Data
(63) Continuation-in-part of application No. 11/732,620, filed on Apr. 4, 2007.
Application No. 11/732,620 is a continuation-in-part of application No. 11/472,607, filed on Jun. 22, 2006, now abandoned.

(51) Int. Cl. A61K 38/49; A61P 7/00

(52) U.S. Cl. 424/94.63

(57) ABSTRACT

A mutant prourokinase plasminogen activator (M5) was developed to make prouPA less subject to spontaneous conversion to tcuPA in blood at therapeutic concentrations. Two-chain M5 was shown to form complexes with C1-inhibitor, which was the principal inhibitor of tcM5 in plasma. The effect of supplemental additions of C1-inhibitor on fibrinolysis and fibrinogenolysis by M5 was determined. Supplemental C1-inhibitor restored the stability of high-dose M5 and prevented fibrinogenolysis but not fibrinolysis, the rate of which was not compromised by the inhibitor. Due to higher dose tolerance of M5 in the presence of supplemental C1-inhibitor, the rate of fibrin-specific lysis reached that achievable by nonspecific fibrinolysis, which is the maximum possible for a plasminogen activator. Plasma C1-inhibitor stabilized M5 in plasma by inhibiting tcM5 which would otherwise greatly amplify non-specific plasminogen activation causing more tcM5 generation from M5. This unusual dissociation of inhibitory effects, whereby fibrinogenolysis and not fibrinolysis is inhibited, has significant implications for improving the safety and efficacy of fibrinolysis. Methods of reducing bleeding and non-specific plasminogen activation during fibrinolysis by administering M5 along with exogenous C1-inhibitor are disclosed.

### RELATED APPLICATIONS

This application is a continuation-in-part of U.S. application Ser. No. 11/732,620, filed Apr. 4, 2007, which is a continuation-in-part of U.S. application Ser. No. 11/472,607, filed Jun. 22, 2006, both or which are herein incorporated by reference.

### BACKGROUND OF THE INVENTION

Existing thrombolytic drugs, used in the treatment of thromboembolic diseases, have limited effectiveness and also carry the risk of hemorrhagic complications. Clinical experience with plasminogen activators has highlighted these problems [Rao, et al., J Amer Coll Cardiol. 11: 1-11 (1988); Fennerty, et al., Chest. 95: 88S-97S (1989)]. Since both the efficacy of fibrinolysis and the risk of bleeding are dose-related, the efficacy of therapeutic thrombolysis has always been handicapped by its hemorrhagic side effects; the latter are related to non-specific plasminogen activation causing plasminemia which degrades certain clotting factors (I, V and VIII).

Currently, most therapeutic thrombolysis in the USA is performed using tissue plasminogen activator (tPA) or one of its derivatives. However, thrombolysis is less effective than primary angioplasty which is rapidly replacing it as the treatment of choice, despite the fact that this high-tech procedure delays treatment and increases cost substantially. Improving the efficacy of current thrombolysis by increasing the dose has not been possible. For example, at a dose of 150 mg of tPA per patient, significantly better coronary thrombolysis was achieved but this was accompanied by an unacceptable incidence of intracranial hemorrhage. This side effect obliged the adoption of the currently used less effective dose of 100 mg [Braunwald, et al., J Amer Coll Cardiol. 9: 467 (1987); Grossbard, J Amer Coll Cardiol. 9:467 (1987)].

Similarly, the other natural plasminogen activator, single-chain urokinase plasminogen activator (prouPA), a proenzyme, induced more effective coronary thrombolysis but at infusion rates which caused plasminemia, converted the fibrin-specific single-chain prouPA to non-specific two-chain uPA (tcuPA) and resulted in bleeding [Meyer, et al., Lancet 1:863-868 (1989)].

The bleeding complications of therapeutic thrombolysis can be related not only to the direct lysis of hemostatic fibrin at a vascular injury site but also to the hemorrhagic diathesis caused by plasminemia, resulting in degradation of clotting factors I (fibrinogen) V and VIII, causing a hemophilia-like state. Fibrinogen is the principal protein constituent of a fibrin clot; clotting factor V is a cofactor in the coagulation system, and clotting factor VIII is an essential clotting factor congenitally absent in Hemophilia A.

At physiological concentrations both tPA and prouPA are referred to as “fibrin-specific” plasminogen activators since they target fibrin-bound plasminogen and spare free plasminogen, so that plasminemia is avoided, the enzymatic activity being confined to the fibrin-clot surface by inhibitors in the plasma. However, this physiologic specificity of tPA and prouPA is compromised at therapeutic concentrations. At therapeutic concentrations the capacity of plasma inhibitors to limit the enzymatic reaction to the clot environment is exceeded.

In the case of the proenzyme, prouPA, at therapeutic concentrations its intrinsic catalytic activity is sufficient to activate plasma plasminogen to plasmin. The plasmin in turn then converts single-chain prouPA to tcuPA, which converts much more plasminogen to plasmin. Since tcuPA is a non-specific plasminogen activator, the fibrin-specificity of prouPA is lost at these concentrations. Thus, prouPA's specificity depends on its plasma stability, which then allows tcuPA and plasmin generation to be confined to the fibrin clot [Pannell and Gurewich, Blood, 67: 1215-1223 (1986)].

At therapeutic concentrations, prouPA is especially vulnerable to non-specific plasmin generation since this results in loss of its proenzyme configuration due to its conversion to tcuPA, a non-specific activator, which, being an enzyme, then amplifies systemic plasmin generation several hundred fold.

This cycle of reactions is initiated by the relatively high intrinsic activity of prouPA. For this reason, a prouPA mutation (M5) with a lower intrinsic catalytic activity was developed. A five-fold reduction in intrinsic activity was achieved by a site-directed single residue exchange on a flexible loop in the catalytic domain (Lys300→His) of prouPA [Liu, et al., Biochemistry 35: 14070-14076 (1996)]. This produced a corresponding degree of improvement in plasma stability or inertness at therapeutic concentrations. Unexpectedly, after activation to two-chain M5 (tcM5), the mutant had a two-chain activity almost twice that of tcuPA [Sun, et al, J Biol Chem. 272: 23818-23823 (1997)], indicating the active catalytic site had also undergone a functional change. U.S. Pat. No. 5,472,692 describes a family of prouPA mutants within the flexible loop, of which M5 is one, and the disclosure is incorporated herein by reference.

M5 induced efficient, fibrin-specific clot lysis in a plasma milieu in vitro and in dogs with venous thromboemboli in which M5 was associated with little bleeding [Liu, et al., Circ Res. 90: 757-763 (2002)] in stark contrast to tPA and prouPA. In a second animal study of M5, a more challenging arterial thrombus was selected and M5 was administered by a bolus/infusion administration modeled on the clinical administration of prouPA or tPA. Blood loss from injury sites measured by a highly quantitative new method, from fresh hemostatic sites was ten-fold higher with tPA than M5 (40 ml vs. 4 ml) suggesting that M5 spared hemostatic fibrin at doses which lysed intravascular fibrin clots [Pannell, et al., Blood. 69: 22-26 (1987)]. This difference in the lytic sensitivities of hemostatic versus intravascular fibrin to M5 could be explained by differences in the mechanisms of fibrin-dependent plasminogen activation by M5 compared with tPA [Gurewich, et al., J Thromb Haemost. 4: 1559-65 (2006)]. Specifically, M5 selectively activates plasminogen on partially degraded (fibrin fragment E) and not on intact fibrin, whereas tPA targets plasminogen on intact fibrin (fibrin fragment D) [Liu, et al., J Clin Invest. 88: 2012-2017 (1991)], which corresponds to hemostatic fibrin.

The so-called “Holy Grail” in therapeutic thrombolysis has been to lyse intravascular clots, which obstruct blood flow without degrading fibrin clots which seal blood vessels and prevent bleeding. That is, a treatment that is able to differentiate “bad” clots from “good” clots. Thus, what is needed are novel and non-obvious compositions and methods for the degradation of intravascular clots, which obstruct blood flow without degrading fibrin clots which seal blood vessels and prevent bleeding.

### SUMMARY OF THE INVENTION

ProuPA can become unstable in plasma at therapeutic concentrations. A mutant form, M5, was developed to make prouPA more stable and less subject to spontaneous activation in plasma during fibrinolysis. The spontaneous activation to tcuPA preempted prouPA-mediated fibrinolysis at therapeutic concentrations and seriously compromised prouPA in clinical trials.

Activation of M5 to tcM5 induced a higher catalytic activity than the activation of prouPA to tcuPA, implicating an active site functional difference. An unusual tcM5 complex in dog and human plasma was found. This unpredicted complex comprised M5 and an inhibitor of the compliment pathway, called C1-inhibitor. The effect of C1-inhibitor on fibrinolysis and fibrinogenolysis by M5 is the subject of this application.

Zymography of plasma samples from some of the dogs in the dose-finding phase of the study showed an unusual inhibitor complex with tcM5. This complex was reproduced in vitro in dog and human plasma in which tcM5 (two-chain M5), but not M5, was incubated. The inhibitor was identified to be C1-inhibitor based on its co-migration with a complex formed with purified C1-inhibitor and Western blotting with specific antibodies. It was postulated that endogenous C1-inhibitor helped confine tcM5 activity to the fibrin-clot environment, thereby limiting non-specific plasminogen activation and sparing hemostatic fibrin in these dogs [Gurewich, et al., (2006), supra]. In the Exemplification section, below, it is shown that C1-inhibitor inhibition of tcM5 was further investigated and its effect on fibrin-specific and non-specific plasminogen activation by M5 was characterized in vitro. The inhibition rate by purified human C1-inhibitor (250 μg/ml, the mean physiological concentration) was about seven-fold faster for tcM5 than for tcuPA (10 μg/ml), and several-hundred fold faster than for tPA, an interaction that was previously reported [Huisman, et al., Thromb Haemost. 73: 466-471 (1995)]. The effect of the C1-inhibitor concentration in plasma on fibrinolysis and fibrinogenolysis (indicative of non-specific plasmin generation) by M5 or prouPA was determined by incubating them in plasma at high concentrations (5 and 10 μg/ml) with and without C1-inhibitor supplementation. Without C1-inhibitor, at the lower dose, rapid lysis of a standard clot suspended in the plasma occurred without fibrinogenolysis. At the higher dose of 10 μg/ml, significantly more rapid clot lysis occurred, but this was accompanied by depletion of all the plasma fibrinogen, indicating plasminemia and tcM5 generation. With supplemental C1-inhibitor (doubling the endogenous concentration), the fibrinogen depletion was prevented, indicating that the stability of M5 was restored. The rate of clot lysis at either dose of M5 was not affected by the C1-inhibitor. Therefore, C1-inhibitor prevented non-specific plasminogen activation, which is responsible for plasminemia and the hemorrhagic state, but did not interfere with fibrinolysis, the desired therapeutic effect of M5. This unpredicted and unexpected interaction between C1-inhibitor and M5 (tcM5) is unique and cannot be reproduced by other plasminogen activators or inhibitors. In addition, due to the higher dose tolerance of M5 when supplemental C1-inhibitor was added, the rate of fibrin-specific clot lysis reached the maximum achievable by nonspecific fibrinolysis (that associated with plasminemia and fibrinogenolysis). Plasma C1-inhibitor stabilized M5 in its proenzyme configuration in plasma by inhibiting tcM5, but the rate of inhibition was insufficient to prevent the activation of fibrin-bound plasminogen by M5, which occurs at a faster reaction rate. Other inhibitors, like plasminogen activator-1 (PAI-1), can also stabilize M5 or prouPA, but they do so at the expense of fibrinolysis, which is simultaneously inhibited. The unusual dissociation of effects by the interaction of C1-inhibitor and tcM5 is unprecedented and has significant implications for improving the safety and efficacy of fibrinolysis.

A number of related aspects are described in detail in the following sections.

### BRIEF DESCRIPTION OF THE DRAWINGS

FIG. 1 shows radioisotope counts per minute (mean and one STD) as a percent of the initial count registered by a gamma probe stationed over the femoral artery segment containing the clot formed in the presence of ¹²⁵I-labeled fibrinogen.

FIGS. 2 (A & B) shows thrombi after lysis. (A) Graph showing relative sizes of the residual thrombi found in the femoral artery segments when they were opened at 90 minutes. Size graded 1+−4+, 1+ representing a small fleck and 4+ a cast of the vessel. (B) Digital photos of the thrombi (arrows) found, along with the excised vessel segments. The two M5 segments with residual thrombi are on the left and the three tPA are on the right. The 4+ thrombus seen in the first of the three tPA segments was non-adherent and emitted little radioactivity and was probably related to rethrombosis.

FIG. 3 shows the number of sites in each dog from which significant (>1,000 ODU or >1.3 ml) bleeding occurred. Blood loss was calculated from a measurement of the hemoglobin shed at each of the 4 sites.

FIG. 4 shows the total blood loss (sum of the 4 wound sites) for M5 (mean ˜4 ml) and for tPA (mean ˜40 ml). 75,000 ODU was equivalent to ˜100 ml whole blood.

FIG. 5 shows representative zymograms of plasma in which either tcM5 or UK (5 μg/ml) were incubated. MW marker kDa's are shown in the first lane and minutes across the top. Both tcM5 and UK appear as higher (˜45 kDa) and lower (˜30 kDa) MW lysis zones. The prominent .about.150 kDa inhibitor complex (consistent with C1-inactivator) was seen within minutes with tcM5, whereas it was delayed and only faintly visible with UK. The second inhibitor complex at .about.110 kDa (consistent with antithrombin) was also more prominent with tcM5 than UK. As shown, there was a correspondingly more rapid loss of free tcM5 activity compared with UK.

FIGS. 6 (A & B) shows studies of tcM5:C1-inactivator complexes in plasma (A) and in mixtures of purified tcM5 and C1-inactivator (B). (A) Zymogram of tcM5 in plasma showing the complex. The lower fainter complex probably represents antithrombin (1). Western blot of this plasma showing the complex with UK antibodies (2). Western of tcM5 (3). Western of the plasma showing the complex with C1-inactivator antibodies (5). (B) Zymogram of tcM5 (1). Zymogram of mixture of tcM5 and C1-inactivator showing complexation (3). Coomassie SDS PAGE of tcM5 (4). Coomassie of tcM5 plus C1-inactivator showing the complex (5). Coomassie of C1-inactivator (6). MW markers (A2, B2, 7).

FIGS. 7 (A & B) shows zymograms of tcM5 (5 μg/ml) (A) or tcuPA (5 μg/ml) (B) incubated in pooled bank plasma for 0-60 min. The inhibitor complex at the top, forming within 5 min with tcM5, corresponds to C1 inhibitor, as evidenced by the last lane (C1I) of an incubation (60 min) mixture of purified C1-inhibitor (250 g/ml) and tcM5 or tcuPA. With tcuPA (B) there is more free enzyme seen and the complex is barely visible, reflecting its slower inhibition rate compared with tcM5 (A). The second plasma inhibitor appearing at ˜115 kDa corresponds to antithrombin, a known inhibitor of tcuPA.

FIGS. 8 (A & B) shows zymograms of 120 min incubation mixtures of 10 μg/ml tcM5 (A) or tcuPA (B) with purified C1-inhibitor (250 μg/ml). The more rapid inhibitor complexation by tcM5 corresponds to the more rapid quenching of activity shown in FIG. 3. The minor lower molecular weight lysis bands seen below the two enzymes correspond to by-products of the plasmin activation of the single chain proenzyme forms.

FIG. 9 shows the kinetics of tcM5 (diamonds) or tcuPA (circles) inhibition by C1-inhibitor from the incubation mixtures shown in FIG. 2. At the time points, uPA activity was measured with chromogenic substrate (S-2444). The points graphed are the means of two experiments (error bars are smaller than the symbols; the R2 of the curves was 0.987 for tcM5 and 0.997 for tcuPA). The data were fitted by computer to a non-linear regression for first order logarithmic decay.

FIG. 10 shows the effect of C1-inhibitor (250 μg/ml) when added to bank plasma on plasminogen preservation in the presence of M5 (10, 15 or 20 μg/ml) or prouPA (10 μg/ml) incubated 4 h in plasma. Plasma plasminogen remaining (% of baseline) is represented on the ordinate axis. As shown, supplementation (+) of the plasma with the inhibitor significantly reduced plasminogen depletion by M5 at all doses but not by prouPA at the dose used.

FIGS. 11 (A & B). (A) shows lysis curves determined from release of D-Dimer from clots in a plasma milieu with (+) or without (−) supplemental C1-inhibitor (250 μg/ml) and containing 5 or 10 μg/ml M5. As shown, the presence of the inhibitor did not attenuate the rate of fibrinolysis. (B) shows fibrinogen concentrations remaining at the end of each clot lysis from 5A expressed as % of baseline. At 10 μg/ml of M5 (10−) there was loss of almost all the fibrinogen, reflecting its degradation. However, with supplemental C1 inhibitor (10+) this did not occur.

FIGS. 12 (A & B). (A) shows activity against C1 esterase chromogenic substrate (Spectrozyme C1-E) (open symbols) by 0-15 μg/ml of tcuPA or tcM5, amount adjusted to give comparable uPA substrate (S-2444) (solid symbols) activity. As shown, tcM5 had less C1 esterase-like activity than tcuPA. (mΔA/min is the change in milli-absorbance units (A₄₀₅) with time, reflecting the rate of conversion of chromogenic substrate to product.). (B) shows a representative reducing SDS-PAGE (Coomassie stained) of a 6 h incubation mixture of C4 (480 μg/ml) with buffer (lane 2), tcuPA (lane 3), tcM5 (lane 4) or plasmin (lane 5) (10 μg/ml each). Molecular weight markers are in lane 1. The positions of the α, β, and γ chains of C4 and of the uPA B-chain (lanes 3 and 4) are shown on the right. A faint band is discernable between the α and β chains of C4 in lanes 3 and 4, consistent with a shift of a trace amount of the α-chain by release of the anaphylatoxin peptide. By contrast, a gross degradative effect, particularly of the α-chain, is seen in lane 5.

FIG. 13 shows curves of clot lysis experiments in a plasma milieu at a high of doses of M5. As shown, the clot lysis curves were very similar at these doses. This was because they are at the maximal rate achievable.

FIG. 14 shows bar graphs showing the mean lysis rate and standard deviation (SD) of 3 experiments each at the range of doses of M5 shown. The mean lysis rate was calculated from the time point at which maximal fluorescence released from the fluorescence-tagged fibrin divided by the time. A dose response up to 15 μg/ml was found after which there was a leveling off of the mean lysis rate indicating that a maximum had been reached.

FIG. 15 shows a bar graph showing the percent (%) fibrinogen remaining in each of the plasma samples corresponding to the clot lysis experiments shown in FIG. 13. The mean values and SD are shown. At each of the two doses no additional C1INH or 250, 500 or 750 μg of C1INH indicated by +, ++ or +++ were added. As shown, without added C1INH all of the fibrinogen was degraded (indicated by the 0), meaning that extensive non-specific plasminogen activation with plasminemia causing fibrinogen degradation, had taken place. When this occurs in vivo there is a generalized hemorrhagic state that is induced associated with extensive bleeding. This undesirable side effect of rapid fibrinolysis is progressively inhibited by C1INH. This is an effect that is unique to M5 and means that with this activator, optimal (i.e., maximal) lysis rates are possible when C1INH is administered up to, for example, a plasma concentration of 1,000 μg/ml.

### DETAILED DESCRIPTION OF THE INVENTION

The present invention relates to a novel method of reducing bleeding during fibrinolysis treatment and improving the rate of clot lysis by allowing a higher dose of the activator (M5) to be used. The method is based on the discovery that C1-inhibitor has the ability to inhibit plasminemia by inhibiting the two-chain prourokinase plasminogen activator mutant tcM5. The inhibition rate of this interaction is sufficient to prevent non-specific plasminogen activation but not fibrin-specific plasminogen activation.

ProuPA is a thrombolytic drug with the undesirable side effect of being vulnerable to spontaneous activation (to tcuPA) in plasma at therapeutic concentrations. M5 is a single site mutant of prouPA developed to limit this non-specific effect and, thereby, to allow the fibrin-specific physiological properties of prouPA to be retained at therapeutic doses. The plasminogen activator, M5, differs from the primary sequence of prouPA by a single amino acid substitution at position 300, where the amino acid Lysine has been replaced by Histidine (Lys 300→His) in a flexible loop region within the catalytic domain of single-chain prouPA. This residue exchange lowered the activator's intrinsic, single-chain activity by five-fold and made it more stable in plasma to a corresponding degree [Liu, et al., (1996), supra]. After plasmin-activation to tcM5, the catalytic activity of tcM5 was found to be higher than that of tcuPA [Sun, et al., J Biol Chem. 272: 23818-23823 (1997)], implicating an unanticipated functional difference in their two-chain catalytic sites as well. The present findings, that C1-inhibitor quenches the activity of tcM5 significantly more effectively than that of tcuPA, are consistent with such a difference. This finding was unanticipated since no differences in inhibition by plasminogen activator inhibitor-1 (PAI-1), the principal inhibitor of uPA and tPA, had previously been found.

C1-inhibitor is previously unknown as a plasma inhibitor of tcuPA but was shown to be a very weak inhibitor of tPA. C1-inhibitor is a 104 kDa serine protease inhibitor with a normal plasma concentration of about 250 μg/ml and a half life of about 28 hours. It is an inhibitor of the complement pathway (inhibits C1) and a deficiency of this protein is associated with a disease called hereditary angioedema. As a result, exogenous C1-inhibitor is administered therapeutically for this condition.

As discussed in the Background of the Invention section, ProuPA is a plasminogen activator that is subject to spontaneous activation in plasma at therapeutic concentrations due to its relatively high intrinsic catalytic activity. Plasmin then converts prouPA into tcuPA. The physiological fibrin-specific mechanism of action of prouPA is lost when non-specific conversion to tcuPA takes place. As a result, the selective activation of plasminogen bound to partially degraded fibrin clots and the sparing of intact fibrin clots is lost. This is important since the latter correspond to hemostatic fibrin (“good” clots), whereas the former correspond to intravascular occlusive fibrin (“bad” clots). M5, being more stable in plasma, retains its proenzyme form at therapeutic concentrations, and thereby spares hemostatic fibrin while lysing intravascular thrombi. However, as with prouPA, there is a dose limit to this selective and fibrin-specific effect. For the present invention, experiments were performed in vitro in human plasma at and in excess of this dose or plasma concentration limit. The effects of M5 were compared with prouPA, its parent molecule, and tPA, currently the most commonly used thrombolytic drug in the USA.

The serine proteases tPA and prouPA are both natural plasminogen activators and both induce fibrin-specific lysis at limited doses by preferentially activating fibrin-bound plasminogen over free plasminogen. However, distinctly different mechanisms are responsible for this phenomenon. Each activator targets a different fibrin-bound plasminogen. The activator tPA is a single-chain enzyme with a high affinity for a specific binding site on fibrin where tPA forms a ternary complex with an adjacent plasminogen [Hoylaerts, et al., J Biol Chem. 257: 2912-2919 (1982)]. This plasminogen is bound to an internal lysine binding site (Lys-157) in the Aα chain of the D-region of fibrin [Nieuwenhuizen W, et al., Biochim Biophys Acta. 748: 86-92 (1983)]. In the presence of fibrin fragment D binding site, which is constitutive of intact fibrin, plasminogen activation by tPA is promoted by as much as 1,000-fold [Petersen, et al., Biochim Biophys Acta. 952: 245-254 (1988)], reflecting the importance of the ternary complex with intact fibrin for tPA.

By contrast, the single-chain prouPA has no fibrin affinity. Yet when a clot is added to plasma containing prouPA (or M5), local activation of a fraction of the prouPA takes place on the fibrin surface and lysis is triggered [Liu, et al., Biochemistry 35: 14070-14076 (1996)]. This sequence of events is facilitated by a conformational change in plasminogen for which prouPA (or M5) has high substrate affinity. This change occurs when plasminogen binds to its carboxy-terminal lysine binding site in the E region of fibrin. In the presence of fibrin fragment E, plasminogen activation by single-chain prouPA/M5 is equal to that of its two-chain derivative, tcuPA or tcM5, corresponding to a several hundred-fold promotion of its intrinsic activity [Liu and Gurewich, Biochemistry 31: 6311-6317 (1992)].

Therefore, tPA and prouPA/M5 induce fibrin dependent plasminogen activation different fibrin-bound plasminogen substrates. In the case of tPA, it is dependent on an internal lysine plasminogen binding site in the fibrin D region, whereas prouPA/M5 is dependent on carboxy-terminal lysine binding in the E region of fibrin. This difference clear cut since there is little or no reciprocity. In a purified system, plasminogen activation by tPA is promoted by fibrin fragment D and that by prouPA/M5 is promoted only by fibrin fragment E [Liu and Gurewich, J Clin Invest. 88: 2012-2017 (1991)]. Newly formed intact (hemostatic) fibrin contains only the internal lysine plasminogen binding site in the D region of fibrin. The carboxy-terminal lysines in the E region are not created until after plasmin degradation has occurred [Harpel, et al., J Biol Chem. 260: 4432-4440 (1985)]. This difference is evidenced by the lag phase which is characteristic of prouPA (or M5)-induced clot lysis in a plasma milieu and by the fact that the lag phase is substantially attenuated by gentle pre-treatment of the clot with plasmin, which creates the fibrin fragment E carboxy-terminal lysine plasminogen binding sites. Conversely, tPA-induced clot lysis in plasma has no lag phase and tPA lyses intact and degraded fibrin clots equally well under these same conditions [Pannell, et al., J Clin Invest. 81: 853-859 (1988)]. Thus, intact fibrin is relatively resistant to lysis by prouPA/M5 whereas it is not resistant to tPA.

Hemostatic fibrin, consistent with its physiological function, is protected from plasmin degradation by several physiological safeguards. These include the inhibition of free tPA (and tcuPA) by PAI-1 and the removal of carboxy-terminal binding sites on fibrin by thrombin-activated procarboxypeptidase in plasma [Hendriks, et al., J Clin Chem. Clin Biochem. 27: 277-280 (1989)]. By contrast, when an intravascular thrombus forms and causes a vascular occlusion, physiological mechanisms for its degradation are triggered. In particular, there is a release of tPA from the vessel wall which, aided by the local stasis, binds to the thrombus. Fibrin degradation is initiated thus creating new carboxy-terminal lysine plasminogen binding sites which facilitate lysis [Harpel, et al., J Biol Chem. 260: 4432-4440 (1985)], particularly by prouPA fibrinolytic paradigm by which M5 functions.

The creation of the new (C-terminal) plasminogen binding sites by fibrin degradation on the fibrin fragment E region of fibrin differentiates the intravascular fibrin thrombus from hemostatic fibrin. This difference provides an explanation for why M5 can induce effective thrombolysis without degrading hemostatic fibrin. However, this selectivity in its targeted action will be lost if M5 becomes unstable and systemic tcM5 generation occurs. The C1-inhibitor in plasma is important to the stability of M5 and its stabilizing effect can be reinforced by augmenting the C1-inhibitor concentration with supplemental exogenous C1-inhibitor. Moreover, this method of reinforcing the fibrin selectivity and safety of therapeutic thrombolysis with M5 can be accomplished without interfering with its thrombolytic efficacy. This is the invention. No other plasminogen activator inhibitor is known to have this effect in which non-specific effects responsible for side effects are inhibited and the therapeutic effects are fully retained. In contrast, the D region ternary complex plasminogen binding site is present in hemostatic fibrin, making it more vulnerable to lysis by tPA. This concept is consistent with tPA-associated bleeding which has been shown to have a low correlation with fibrinogen degradation, a sign of non-specificity [Montoney, et al., Circulation. 91: 1540-1544 (1995)]. This is because this bleeding is related to direct lysis of fibrin at a hemostatic site. By contrast, bleeding with prouPA is directly correlated with non-specificity, which leads to a generalized hemorrhagic state.

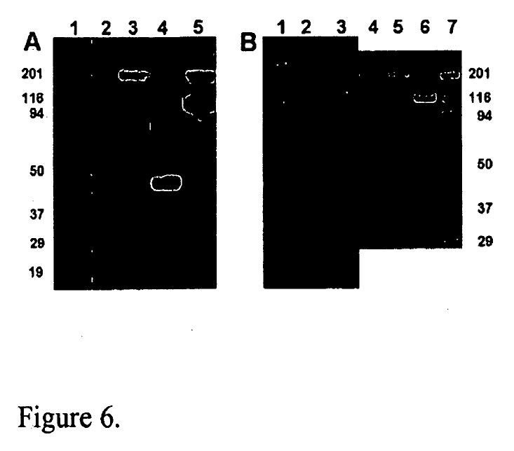

The present invention concerns the effect of C1-inhibitor on fibrinolysis and fibrinogenolysis by M5. As discussed in the Exemplification section which follows, C1-inhibitor was the predominant inhibitor complex with tcM5 in plasma (FIG. 7A), but only a minor inhibitor of tcuPA, suggesting that C-1 inhibitor helps prevent non-specific plasmin generation by M5/tcM5, an unexpected effect not seen with other plasminogen activators. During thrombolysis, plasmin generation must be confined to the fibrin clot environment. If not, plasminogen in the ambient plasma will be activated and the proenzyme (M5 or prouPA) converted to the two-chain enzyme. It is the effect of certain inhibitors in plasma which confine tcM5 or tcuPA activity to the clot environment. Congenital inhibitor deficiencies are associated with bleeding. Prior to this invention plasma C1-inhibitor had not been identified to function in this capacity and indeed it is only a very weak inhibitor of the natural plasminogen activators, tcuPA or tPA. By contrast, the present invention shows C1-inhibitor it is sufficiently reactive to tcM5 to prevent non-specific plasminogen activation by M5. Moreover, C1-inhibitor is an abundant plasma inhibitor (˜250 μg/ml) in contrast to PAI-1 (the principal inhibitor of tcuPA or tPA) which is present physiologically only in ng quantities only.

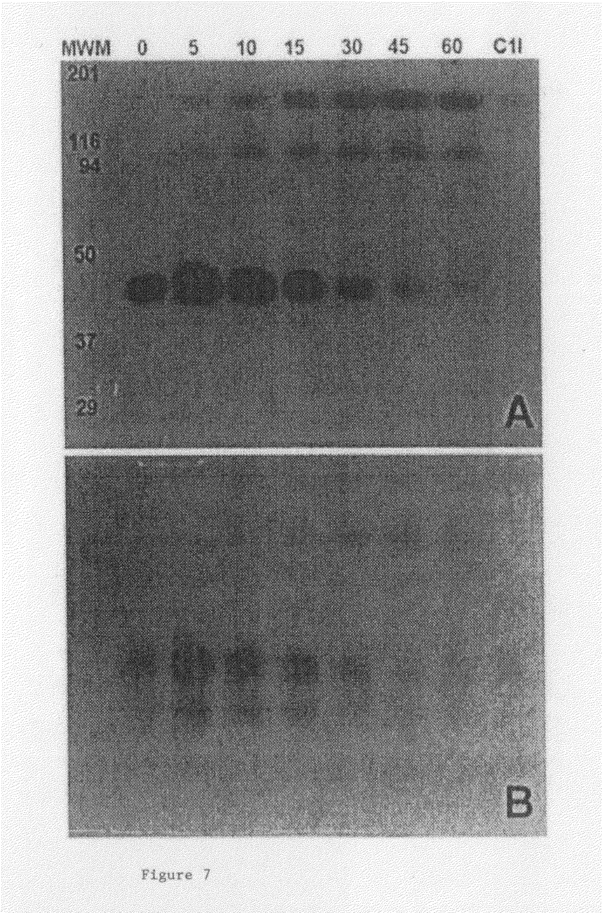

As discussed in the Exemplification section, when tcM5 and tcuPA were incubated with exogenous C1-inhibitor in plasma. Zymograms of the plasma showed a rapid and progressive loss of the tcM5 free enzyme associated with the appearance of prominent inhibitor complexes (FIG. 8A). Relatively little inhibition of tcuPA by C1-inhibitor was seen (FIG. 8B). There was essentially complete inhibition of tcM5 within 60 min, whereas 30% of the tcuPA activity remained even after 3 h of incubation. In a purified system, the inhibition rate of C1-inhibitor was about seven-fold greater against tcM5 than tcuPA. As shown in Table 1, the inhibition of tcuPA was about 100-fold faster than that previously described for tPA. The inhibition of tcM5 in plasma is what limits the cyclic reactions which would otherwise result in plasmin generation and lead to more tcM5 and plasmin generation, and bleeding. The efficient inhibition of tcM5 by C1-inhibitor can, therefore, contribute significantly to confining tcM5 generation to the intravascular clot environment, thereby protecting the more remote hemostatic fibrin.

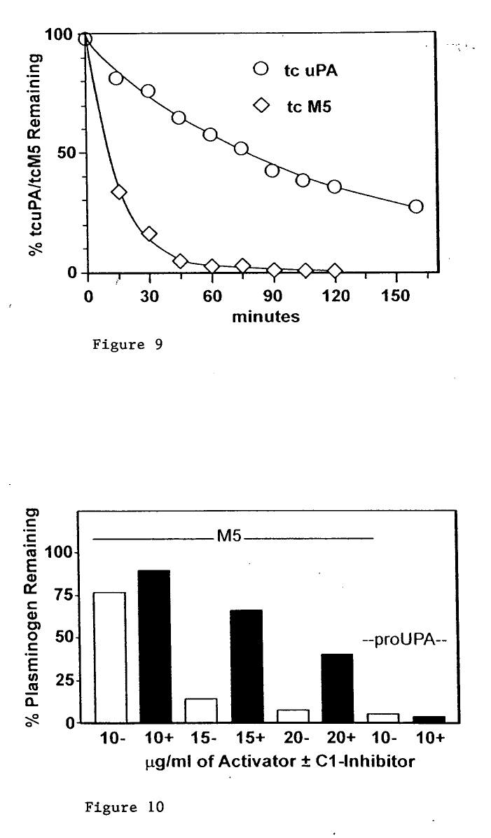

As discussed in the Exemplification section, when plasma was supplemented with C1-inhibitor (250 μg/ml), the M5 concentration threshold at which tcM5 conversion and non-specific plasminogen activation occurred was substantially raised from 5 to 10 μg/ml (FIG. 10). Therefore, C1-inhibitor, by inhibiting tcM5, effectively and unexpectedly helped stabilize M5 in plasma and allowed a higher concentration of M5 to be tolerated without compromising fibrin-specificity. By contrast, as shown by the last two bars in FIG. 10, C1-inhibitor supplementation had no attenuating effect on plasminogen activation of prouPA at the high concentrations used in these experiments.

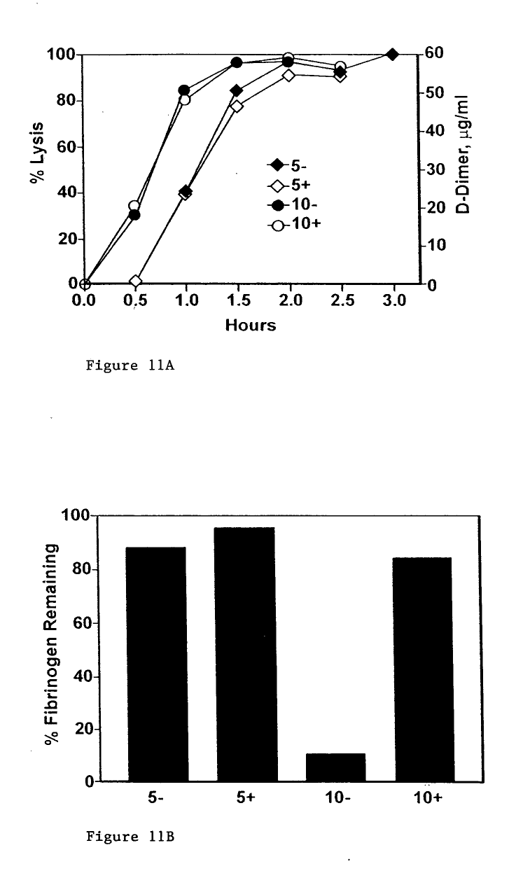

When fibrin-dependent plasminogen activation was tested by measuring the rate of clot lysis by M5, no inhibition of fibrinolysis by supplemental C1-inhibitor was observed (FIG. 11A). However, fibrinogenolysis, reflecting non-specific plasminogen activation, was prevented (FIG. 11B). This difference between the inhibition of non-specific plasminogen activation (fibrinogenolysis) and fibrin-dependent plasminogen activation (fibrinolysis) was unexpected and unprecedented. It could not have been anticipated from previous studies with prouPA or tPA, and supplemental PAI-1 showed that this inhibitor inhibited both reactions (fibrinogenolysis and fibrinolysis) to a comparable degree.

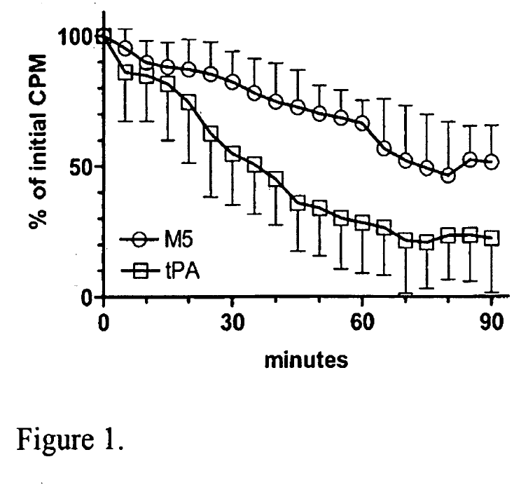

These results of the present study make clear, and the present invention includes, methods of administering an amount of exogenous C1-inhibitor along with M5 during fibrinolysis treatment that limit non-specific plasminogen activation in the blood and reduce bleeding complications of thrombolytic therapy. An amount of M5 sufficient to cause rapid lysis of an occlusive blood clot should be administered along with an amount of exogenous C1-inhibitor sufficient to limit non-specific plasminogen activation in blood. Moreover, since C1-inhibitor permits higher doses to be safely administered, faster and even maximal rates of lysis are possible, as shown in FIG. 1A. The M5 mutant is administered as a thrombolytic agent in the same way as prouPA, or tPA. M5 is mixed with a pharmaceutically acceptable carrier, e.g., saline, and administered by intravascular, e.g., intravenous or intra-arterial route. The C1-inhibitor is administered, for example, as a bolus intravascularly just prior to or at essentially the same time as the M5. The present invention includes methods where M5 may be injected as a bolus of approximately 20 to 60 mg, or may be infused intravenously at a rate of 100-300 mg/hour. Another embodiment, the present invention includes a method of inhibiting the enzymatic activity of tcM5. As is detailed in the Exemplification, an amount of exogenous C1-inhibitor sufficient to limit the systemic formation of tcM5 from M5 activation should be administered to a patient desiring to limit the activity of tcM5. It should be noted that C1-inhibitor can be administered prior to or simultaneously with M5. Likewise, both C1-inhibitor and M5 can be administered slowly (for example, via a drip i.v., or the like) or via a “high infusion rate” procedure such as via an i.v. bolus or the like. Use of high infusion rate administration result in what can be termed “high infusion rate fibrinolysis.”

In another embodiment, the present invention includes a method of increasing the plasma stability or inertness of M5 during fibrinolysis treatment. M5's stability is increased by administering exogenous C1-inhibitor sufficient to establish a C1-inhibitor concentration within the range of 1.5 to 4, preferably 2 to 3, times the mean physiological concentration of C1-inhibitor. The C1-inhibitor inhibits non-specific plasminogen activation by tcM5 without inhibiting fibrinolysis medicated by tcM5.

The present invention includes the treatment of a patient with M5 and supplementing the endogenous C1-inhibitor concentration if it is found to be low. The average (mean) physiological level is about 250 μg/ml, but the range is from 150-450 μg/ml. Individuals at the lower end of the range, especially those being treated for ischemic stroke, would be at a higher risk of bleeding from therapeutic concentrations of M5 so their need for supplementation would be greater and should brought up to at least 250 μg/ml. The concentrations established in plasma during treatment should be within the range of about 1.5 to about 4, preferably about 2 to about 3, times that of average physiological levels or approximately 0.5 g/l (grams/liter) to 1.5 g/l. As is detailed in the Exemplification section, a C-1 inhibitor supplementation of 0.25 g/l was effective in promoting the plasma stability of M5, and double the M5 threshold concentration (to 10 μg/ml), a level at which non-specific plasminogen activation could otherwise have occurred.

At the same time that M5 fibrinolysis remained unimpaired fibrinogenolysis was inhibited by supplementation with C1-inhibitor. Therefore, C1-inhibitor restored fibrin-specificity at a more rapid, and even maximal clot lysis rate which otherwise would not be possible without inducing non-specific plasminogen activation and hemorrhagic side effects. That is to say, C1-inhibitor raises the dose-limit for fibrin-specific clot lysis by M5 to the point at which it is maximal for a plasminogen activator. In the presence of supplemental C1-inhibitor, the rate of fibrinolysis by M5 becomes equivalent to that obtained at maximum non-fibrin specific doses, which is the maximal rate. All plasminogen activators lyse fibrin indirectly, since it is plasmin which degrades the fibrin. Therefore, the maximal rate possible by a plasminogen activator is that at which plasmin degradation of fibrin becomes the rate limiting step. C1-inhibitor supplementation made possible a maximal fibrinolysis rate by M5 in vitro without sacrificing fibrin-specificity. Therefore, the present invention also includes methods of accelerating the rate of therapeutic fibrin clot lysis safely, without risking a hemorrhagic side effect. The methods include administering M5 in an amount sufficient to establish concentrations of about 10-15 μg/ml in plasma, so that fibrinolysis becomes maximal, together with exogenous C1-inhibitor in an amount sufficient to establish a concentration within the range of about 1.5 to 4, preferably about 2 to 3, times mean physiological plasma concentration or about 375-750 μg/ml.

The methods of the present invention include the administration of exogenous C1-inhibitor mixed with a pharmaceutically acceptable carrier and administered as a bolus prior to the administration of M5, wherein the level of C1-inhibitor in the plasma of the patient is brought into the high physiological range. Included is the method wherein the level of C1-inhibitor established in the plasma of the patient is about 500-750 μg/ml, 2-3 times the mean physiological concentration. The methods include administering the C1-inhibitor as a bolus injection prior to thrombolysis with M5. The half-life of native C1-inhibitor is about 28 hours. That of recombinant C1-inhibitor can be as short as three hours. The appropriate amount of exogenous C1-inhibitor to administer will depend on its half-life. Plasma-derived C1-inhibitor is currently used to treat hereditary angioedema and a recombinant form has been submitted for approval. For the purpose of thrombolysis with M5, the longer half-life of native C1-inhibitor is not necessary because the C1-inhibitor is needed for no more than 90 minutes. Therefore, a recombinant or transgenic C1-inhibitor, with a shorter half-lives may be used, including that made in yeast or E. coli. Recombinant forms may be non-glycosylated or glycosylated differently than the heavily glycosylated native C1-inhibitor. The differences in glycosylation affect catabolism and may affect antigenicity. These factors may pose problems for the hereditary angioedema indication for which repeat injections are required and a long half-life is preferable. However, neither of these are significant considerations for the thrombolysic indication, which is usually a one-time treatment of short duration. C1-inhibitor enzymatic inhibition is determined by the protein rather than its glycosyl moiety. Therefore, both the native and the recombinant forms of C1-inhibitor are suitable to allow maximal rates of thrombolysis and optimal fibrin specificity and minimal bleeding. Therefore the present invention includes methods wherein the exogenous C1-inhibitor administered is of recombinant origin, including a chimera, or of native origin. The methods include the case wherein the exogenous C1-inhibitor is non-glycosylated or differently glycosylated from the native form.

C1-inhibitor also had a modest effect on non-specific plasminogen activation by prouPA but not at therapeutic concentrations of prouPA. The rate of inhibition of tPA by C1-inhibitor is even less, as previously reported [Huisman, et al., Thromb Haemost. 73: 466-471 (1995)]. The principal plasma inhibitor of tPA and tcuPA is plasminogen activator inhibitor-1 (PAI-1). However, increased levels of PAI-1 have been associated clinically with impaired fibrinolysis [Juhan-Vague, et al., Thromb Res. 33: 523-530 (1984); Meade, et al., Lancet. 342: 1076-1079 (1993)] and in vitro supplementation (25-100 ng/ml) of plasma with PAI-1 inhibits clot lysis along with fibrinogenolysis (unpublished observations). Therefore, the differential inhibition of fibrinogenolysis but not fibrinolysis by M5 in the presence of C1-inhibitor was unexpected and novel. These previous findings with PAI-1 are in stark contrast to the present findings with C1-inhibitor, wherein clot lysis was not inhibited.

C1-inhibitor complexes were identified by the inventors. It was postulated that this inhibition of tcM5 by endogenous C1-inhibitor contributed to the ten-fold lower blood loss associated with M5 compared with tPA [Gurewich, et al., (2006), supra]. The present findings demonstrate the unpredicted finding that if supplemental C1-inhibitor had been administered to these dogs, faster lysis rates from higher doses accompanied by the same low bleeding incidence would have been achieved.

The present invention includes a method of preventing side effects, such as bleeding, during fibrinolysis with M5 by administering exogenous C1-inhibitor just before, simultaneously with or essentially simultaneously with M5. The M5 is administered in an amount sufficient to cause a maximal rate of lysis of an occlusive blood clot and the exogenous C1-inhibitor is administered in an amount sufficient to limit non-specific plasminogen activation by M5. The maximal rate of lysis is the rate at which the plasmin degradation of fibrin (the final step in fibrinolysis rather than the plasminogen activation by M5) is the rate limiting step in clot lysis. This occurs at a plasma M5 concentration of ˜10 μg/ml. Moreover, since it is fibrin-bound plasmin which induces fibrinolysis, rather than the free plasminogen in plasma, non-specific plasminogen activation (plasminemia) has no useful function. It only produces side effects. Using supplemental C1-inhibitor supplementation, the activation rate of fibrin-bound plasminogen by M5 can be made optimal without risking non-specific plasminogen activation and its attendant hemorrhagic and other side effects. The methods of the present invention include methods of increasing the dose-range of fibrin-specific lysis by M5 in a patient. Included methods comprise administering exogenous C1-inhibitor in an amount sufficient to prevent non-specific plasminogen activation by M5. Included is the method wherein the exogenous C1-inhibitor administered establishes a concentration of C1-inhibitor, in the plasma of the patient that is within the range of about 1.5 to 4, preferably about 2 to 3, times the mean physiological level.

The dissociation observed between the inhibition of nonspecific and fibrin-specific plasminogen activation by C1-inhibitor implicates two different rates of plasminogen activation. This is, in fact, well-known to be the case. Fibrin bound plasminogen is activated more rapidly than free native plasminogen in plasma. In the case of prouPA or M5 plasminogen activation is promoted more than two-hundred fifty fold by fibrin, specifically fibrin fragment E [Liu, et al., Biochemistry 31: 6311-6317 (1992)]. The inhibition rate of tcM5 by C1-inhibitor falls well between these two rates, whereas this is not the case with other anti-activators. Nor does this differential inhibition apply to other known plasminogen activators.

In conclusion, C1-inhibitor was unpredictably much more reactive against tcM5 than tcuPA and, when added to plasma, it prevented non-specific plasmin generation by M5 at high fibrinolytic concentrations. Since plasminemia can cause bleeding [Rao, et al., (1988), supra; Fennerty, et al., (1989), supra], clotting [Hoffmeister, et al., Thromb Res. 103: S51-S55 (2001)], the “plasminogen steal” phenomenon [Torr, et al., J Amer Coll Cardiol. 19: 1085-1090 (1992)], and complement activation [Bennett, et al, J Amer Coll Cardiol. 10: 627-632 (1987)], limiting non-specific plasmin generation without interfering with fibrinolysis is of special clinical interest. An inhibitor that controls this dose-related plasminogen activator side effect has the potential to optimize the lysis rate and minimize side effects. This invention helps overcome two major limitations of therapeutic thrombolysis, which have limited the use of this therapeutic modality. These are its inadequate efficacy and its high complication rate, particularly major bleeding. The unpredicted use of C1-inhibitor to improve both of these important limitation of therapeutic thrombolysis is possible only with the plasminogen activator, M5.

### EXEMPLIFICATION

### Example

### Clot Lysis In Vivo

Materials

Recombinant Lys300→His proUK expressed in Escherichia coli was prepared as previously described [Liu, et al., Circ Res. 90: 757-763 (2002)] and obtained from Primm (Milan, Italy). Single-chain tPA, pharmaceutical grade, was purchased from Genentech (San Francisco, Calif.). Recombinant proUK expressed in E. coli was obtained from Landing Science and Technology Company, Nanjing, China. Aprotinin was obtained as Trasylol from Miles, Inc., Kankakee, Ill. Purified human C1-inactivator was obtained from ZLB Behring, Germany.

Methods

Fibrinogen was measured as thrombin clottable protein. Plasma (0.5 ml) was diluted with 2 volumes of 0.06 M sodium phosphate, pH 6.1. One volume of thrombin (100 NIH units/ml; ThromboMax from Sigma, St. Louis, Mo.) was added and mixed and incubated for 30 min at 37° C. The clot was wound onto a wooden stick to express the diluted serum proteins, rinsed by standing in 5 ml of the buffer; then deposited into a tube with 1 ml of 5% NaOH. After boiling for 1 min, the clot was dissolved and the protein was measured spectrophotometrically at 280 nm.

Laemmli SDS-PAGE electrophoresis was carried out in 10% polyacrylamide slab gels. Zymography was performed according to the method of Granelli-Pipemo and Reich (Granelli-Piperno and Reich, J Exp Med. 148: 223-234 (1978)) as modified by Vassalli, et al., (Vassalli et al., J Exp Med. 159: 1653-1658 (1984)). After electrophoresis, the polyacrylamide slabs were washed by agitation for 2 hours in 2.5% Triton X-100 in water, followed by 1 hour in 0.1 M Tris-HCl (pH 8.0), and then layered over an underlay consisting of 0.8% agarose (Agarose low melting, Fisher Biotech), casein (2% w/v; Carnation Non-fat Dry Milk), and plasminogen (20 μg/ml) in 0.1 M Tris-HCl (pH 8.0). With incubation the electrophoretic bands of plasminogen activator produced a cleared zone in the white casein background. Inhibitor complexes become active in this system.

For western blotting, proteins were transferred to nitrocellulose membrane (Amersham Biosciences) and probed with specific antibodies to urokinase (American Diagnostica) and to C1-inactivator (ZLB Behring, Germany) and developed with the Pierce Supersignal West Dura Kit.

In Vivo Studies

All procedures in animals were in accordance with the Guide for the Care of Animals (National Academy of Science, 1996) and were approved by the Animal Studies Committee at the University of Pittsburgh, McGowan Institute of Regenerative Medicine.

Thrombolysis Animal Model

Dogs were chosen as the experimental animal for these studies because of the well-established species specificity of proUK/UK. Dogs are one of the few animals comparably sensitive to the human enzyme as man. The animal model of arterial thrombosis described by Badylak, et al., [J Pharmacol Methods 19: 293-304 (1988)] and previously used to evaluate proUK [Badylak, et al., Thromb Res. 52: 295-312 (1988)] was used for M5 in this study. All the animal experiments were performed at the University of Pittsburgh. In brief, female beagle dogs weighing 7-10 kg were anesthetized with pentobarbital and maintained at a surgical plane of anesthesia with isoflurane. The left femoral artery, with the associated profunda branch, was isolated. The profunda femoris branch was cannulated (PE 0.5 mm ID) to provide access to the segment. Proximal and distal ligatures were placed in order to delineate a 1.5-2 cm segment of the vessel. After extracting all blood from the segment using a syringe, the segment was filled with hot (>90° C.) saline for 5 minutes. After a 5-minute exposure, the saline was removed, and blood flow restored through the segment for 20 seconds, following which the segment was allowed to fill with blood by retightening the distal ligature followed by the proximal. A tracer of 15 μCi of ¹²⁵I-labeled fibrinogen (Amersham Corp., Arlington Heights, Ill.) was then instilled into the segment through the access branch and thoroughly mixed, followed by 100 units of thrombin (Sigma, St. Louis, Mo.) in 0.05 ml saline. At the end of 15 minutes, the proximal ligature was opened allowing some contact with the circulation, and at the end of 30 minutes, the time needed for the clot to become fully adherent to the vessel wall, the distal ligature was opened in preparation for the infusions.

The radioactivity over the thrombus was monitored continuously for 90 minutes with a ¹²⁵I-specific gamma probe (Eberline Co., Santa Fe, N. Mex.) positioned over the femoral artery segment. After 90 minutes, the segment was isolated by double ligatures at each end, removed by cutting between the ligatures, and its contents examined after opening and spreading the vessel. Residual clot was graded 1+−4+ with 1+ representing one or two small flecks and 4+ a larger clot filling the segment. The open vessel and its contents were photographed with a digital camera.

Infusion of M5 or tPA

Dose finding experiments in this model were first undertaken in order to establish the dose of each activator which was both effective and relatively fibrin-specific, defined as fibrinogen consumption of <40%. Doses of 2 mg/kg M5 and 1.4 mg/kg tPA were selected on that basis, of which 20% was administered as a bolus by push with a 10 ml syringe and the remainder by constant infusion over 60 minutes with an infusion pump (Harvard). Compared with the previous study [Liu, et al., Circ Res. 90: 757-763 (2002)], the dose of M5 in the present study was about 40% less, whereas the tPA dose was 40% more. Each activator was made up in a solution containing 1 mg/ml and administered via a catheter in the jugular vein of the dog. Twelve dogs were infused alternatively with M5 or tPA. Significant endogenous lysis in this model does not occur in 90 minutes [Badylak, et al., J Pharmacol Methods 19: 293-304 (1988); Badylak, et al., Thromb Res. 52: 295-312 (1988)]. Therefore, a placebo group was not included.

Experimental Model of Rebleeding

In each animal, two pairs of previously standardized injuries were made and evaluated during the dose-finding stage of this study. Over the right and left sides of the shaved upper abdomen of the anesthetized dog, a 1 cm² skin-deep incision was made from which the epidermis was peeled off as previously described [Liu, et al., Circ Res. 90: 757-763 (2002)]. One of the exposed small vessels in the dermis at each site was cut until bleeding ensued and then dabbed at intervals until hemostasis.

Secondly, after removing the hair from the dorsal surface of each ear, a full thickness incision was made 3-5 mm in length using a #11 scalpel blade in an area devoid of visible vessels, as previously described in rabbits [Marder, et al., Thromb Res. 67: 31-40 (1992)]. The bleeding points on both surfaces of each ear were dabbed at intervals until hemostasis.

Administration of M5 or tPA was not started until bleeding at all four sites had ceased. When rebleeding occurred during the infusions, the blood was absorbed into gauze pads over the 90-minute duration of the experiment. The gauze pads from each of the bleeding sites were collected separately into plastic bags and analyzed the following day as follows: The gauze pads were placed in a measured amount of distilled water to hemolyze the red cells allowing the hemoglobin to go into solution (the gauze returned to its original white in the water). The hemoglobin concentration was then measured by spectrophotometry at 410 nm (in optical density units, ODU). For a hematocrit of 40-45%, which was average for these dogs, 75,000 ODU corresponded to about 100 ml whole blood.

Blood Sampling

Blood samples were collected from the jugular vein contralateral to the one used for the infusion. Samples were collected into tubes containing citrate (1:9) and aprotinin (200 KIU/ml final concentration) and were obtained at baseline, 55 minutes and 90 minutes.

Fibrinogen concentrations were measured in all samples and expressed as a percent of the baseline value. The 55-minute sample was also used for zymography to evaluate inhibitor complexes and to estimate the M5 concentration.

Incubation of tcM5 and UK in Plasma

The stability of M5 or proUK in plasma depends on the efficiency by which tcM5 or UK is inhibited. Without inhibitors, a mixture of M5/proUK and plasminogen will spontaneously convert to tcM5/UK and plasmin, though this does occur less rapidly with M5 than with proUK. Therefore, the efficiency of inhibition of tcM5 by plasma inhibitors is relevant and was evaluated in comparison with UK in dog and human citrate plasma. M5 and recombinant proUK from E. coli were each activated with plasmin by the method of Pannell and Gurewich [Pannell and Gurewich, Blood 1987; 69: 22-26 (1987)]. The kinetics of plasmin activation of M5 and proUK are comparable. Each enzyme (5 μg/ml) was then incubated (37° C.) in citrate plasma (human or dog) and samples removed for zymography at time intervals for 60 minutes. The experiments were repeated several times using different sampling intervals. The in vitro zymograms were compared with zymograms obtained from the 55-minute samples from certain dogs in the M5 dose-finding study in which inhibitor complexes were detectable (higher doses).

Statistics

Statistical analysis was by the Mann Whitney two-tailed test using GraphPad Prism version 3.03 for Windows, GraphPad Software, San Diego, Calif. USA.

Results

Lysis of the Femoral Artery Thrombus

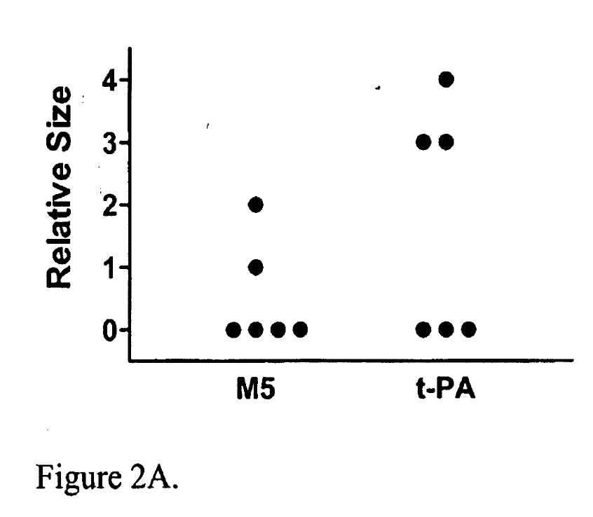

A gradual decline in radioactivity recorded over the segment occurred during the infusions with each activator. After 90 minutes, the radioactivity reached about 20% of baseline with tPA and about 50% with M5 (FIG. 1), suggesting superior lysis by tPA. However, this was found not to be the case when the segments were opened and examined. In 3 of the tPA dogs, large clots (3-4+) were found, of which one was due to rethrombosis (see below). In the M5 dogs, 2 residual thrombi were found, which were smaller (1-2+) (FIGS. 2A & 2B). These results were similar and indicated that clot lysis by the two activators was comparable.

In the tPA dog with a 4+ clot, a cast of the segment (2B, first tPA photo), the clot was non-adherent whereas residual thrombi were invariably tightly adherent. This suggested it was due to rethrombosis, a conclusion consistent with its negligible radioactivity.

The discrepancy between the radioisotope and anatomic findings with M5 indicated that in the 5 dogs with little (one) or no (four) thrombi, the radioactivity was found to come from the vessel wall and segment bed. Why this diffusion of radioactivity during lysis occurred more with M5 than tPA remains to be explained.

The addition of a flow probe as a substitute was attempted but abandoned because it required frequent adjustments which interfered with keeping the isotope monitor in position.

Rebleeding from the Four Injury Sites

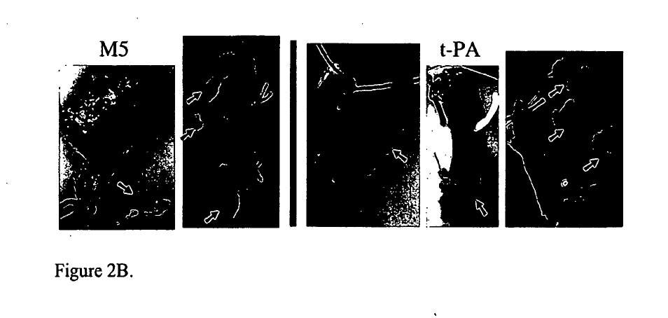

Rebleeding was defined as blood loss at a wound site>1,000 ODU (˜1.3 ml) [Note, the ˜ (tilde) symbol stands for the word “about”]. Rebleeding with tPA tended to occur at multiple sites per dog, being at 3 or more sites in four dogs, 2 sites in one, and 0 sites in one (mean 2.7 sites). Rebleeding with M5 occurred at 3 sites in only one dog and at 0 or 1 site in the remainder (mean 1.2 sites) (p=0.062) (FIG. 3).

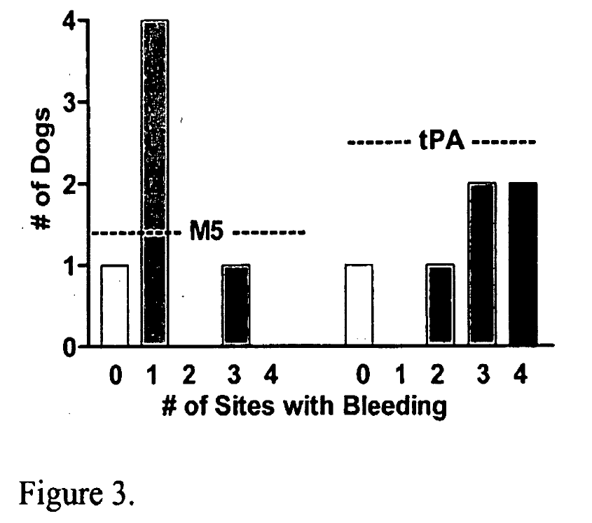

Total blood loss (all sites combined) averaged>30,000 ODU (˜40 ml whole blood) in the tPA dogs compared with <3,000 ODU (˜4 ml) in the M5 dogs (p<0.026). Four of the tPA dogs bled extensively with one almost exsanguinating (>80,000 ODU or >110 ml whole blood) (FIG. 4).

Blood Analyses from the Dog Samples

At 55 minutes, the fibrinogen concentrations (mean and range), expressed as a percent of the baseline value, were 60% (47-85) for tPA and 82% (58-100) for M5. At 90 minutes they were similar, being 66% (46-100) and 82% (46-98) for tPA and M5 respectively. The differences between the tPA and M5 fibrinogen values were not statistically significant.

Zymography of the 55-minute samples alongside a range of M5 concentrations indicated the mean M5 plasma concentration in the dogs during the infusion to be about 8 μg/ml (data not shown).

Zymograms of tcM5 and UK Incubated in Plasma

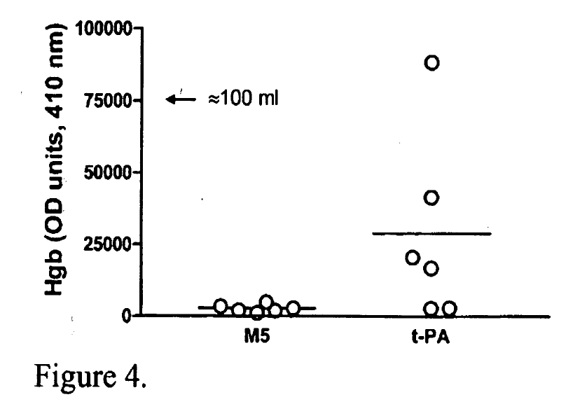

Zymograms of equal concentrations of tcM5 or UK incubated in vitro in either human or dog plasmas showed that both tcM5 and UK appeared as higher (˜45 kDa) and as lower (˜30 kDa) molecular weight bands of activity. The lower band is a more degraded by-product of plasmin-activation of their respective single chain forms routinely seen by this method. In addition, two inhibitor complexes appeared within minutes of incubation of tcM5 in plasma. The position of the predominant inhibitor complex with tcM5 was consistent with C1-inactivator (˜150 kDa) and the lower, less prominent band with an antithrombin complex (˜110 kDa), which is a known inhibitor of UK. These complexes appeared later and were far less prominent with UK. C1-inactivator has not been listed among the known plasma inhibitors of UK [Murano, et al., Blood 55: 430-436 (1980)]. A corresponding more rapid loss of the free enzyme activity bands at 45 and 30 kDa was also seen with tcM5 compared with UK. Complexes with PAI-1 were not visible, probably due to the overwhelming concentrations (5 μg/ml) of the activators. Despite the presence of high molecular weight (HMW) and low molecular weight (LMW) forms of the free enzymes, the inhibitor complexes appeared as single bands of activity, indicating non-resolution of the two forms. These findings were reproducible and comparable in human and dog plasma. A representative zymogram from an experiment in human plasma is shown (FIG. 5).

Zymograms from plasma samples obtained in vivo from the six M5 dogs showed only a band at ˜45 kDa with no detectable complexes, indicating little systemic tcM5 generation (consistent with the little fibrinogen degradation). However, when samples were examined from higher doses used during dose-finding and in which a significant non-specific effect occurred, inhibitor complexes comparable to those seen in the in vitro studies were seen (data not shown).

C1-Inactivator: tcM5 Complex Identification

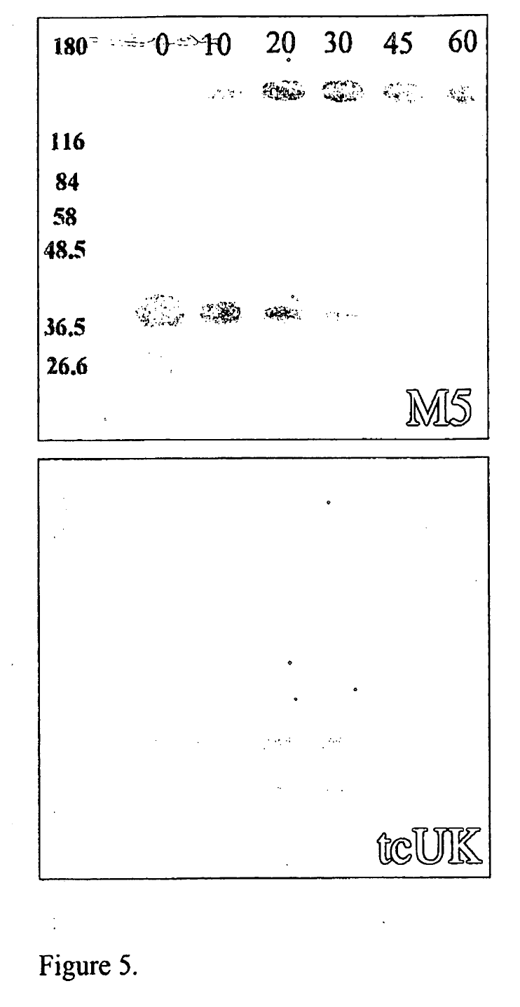

Western blotting with antibodies to UK (FIG. 6A, lane 3,4) and C1-inactivator (A, lane 5) of plasma samples containing tcM5 revealed complexes migrating in the same position with each other and with the predominant complex in a zymogram of this plasma (A, lane 1). However, control plasma showed a C1-inactivator complex in a similar position, probably with factor X11a (not shown). Since the two complexes could not be satisfactorily resolved, studies in a purified system with C1-inactivator and tcM5 were done. A zymogram of tcM5 (B, lane 1) and a mixture of tcM5 and C1-inactivator (B, lane 3) revealed that a complex formed which migrated in the same position as that seen on the plasma zymograms. Similarly, Coomassie staining of SDS PAGE of tcM5 (B, lane 4), tcM5 plus C1-inactivator (B, lane 5), and C1-inactivator (B, lane 6) showed the same enzyme:inhibitor complex. The faint band below C1-inactivator in B lane 5 probably represents a tcM5 antithrombin complex. MW markers are shown in lanes A2, B2 and 7.

Discussion

In a previous in vivo study, M5 caused effective lysis of lung clots in dogs with little bleeding from two hemostatic sites, suggesting that hemostatic fibrin was spared by M5 [Liu, et al., Circ Res. 90: 757-763 (2002)]. Due to the unusual nature of this effect and its potential clinical application, a critical reexamination was conducted with the following protocol modifications: an arterial thrombus was substituted, more injury sites were created, blood loss was precisely quantified by a novel technique, M5 dosage was reduced and tcM5 inhibition in plasma was studied.

Examination of the femoral artery segments at 90 minutes (30 minutes post-infusion) showed effective lysis by M5 with small (1-2+) adherent residual thrombi in only two out of six dogs. In the tPA dogs, there were thrombi (3-4+) in three dogs. However, one of these (4+ thrombus) was due to rethrombosis, based on its non-adherence to the vessel wall and negligible radioactivity. Therefore, the efficacy of thrombolysis by M5 and tPA was comparable. Although in the previous dog study clot lysis by M5 was superior to tPA, the M5 dose in that study was 40% higher and the tPA dose 40% lower [Liu, et al., Circ Res. 90: 757-763 (2002)].

The radioisotope counts over the segment suggested that lysis was slower and less complete with M5. The discrepancy between this surrogate endpoint and the above findings indicated that in the five M5 animals with little or no residual thrombus (FIG. 2B), the radioactivity came from elsewhere and it was found to emanate from the vessel wall and surrounding tissue. Why this occurred more with M5 than tPA remains to be determined. In addition, due to rethrombosis in one tPA dog, the radioisotope findings were not a reliable indicator of the vessel lumen content for either activator in this study.

Due to the small size of this study, little significance can be attached to the finding of rethrombosis in one of the tPA animals. However, this event is consistent with previous studies in tPA-treated dogs [Rapold, et al., Blood 77: 1020-1024 (1991)], with clinical studies in which a 25-30% early coronary reocclusion incidence was reported with tPA [Wilson, et al., Am Heart J. 141: 704-710 (2001)], and with reports that the efficacy of percutaneous coronary intervention over tPA was related to a higher reocclusion rate with tPA [Armstrong, et al., Circulation 107: 2533-2537 (2003)]. By contrast, coronary reocclusion rates of only 0-5% were reported with proUK [Pannell and Gurewich, Blood, 67: 1215-1223 (1986); Weaver, et al., J Am Coll Cardiol. 24: 1242-1248 (1994); Zarich, et al., J Am Coll Cardiol. 26: 374-379 (1995)], and markers of thrombin generation in plasma were not induced by proUK [Weaver, et al., J Am Coll Cardiol. 24: 1242-1248 (1994)], in contrast to tPA [Owen, et al., Blood. 72: 616-620 (1988)].

In conclusion, M5 is a single site mutant of proUK with a lower intrinsic activity and thereby superior stability in plasma, which enables its pro-enzyme fibrinolytic properties to be better preserved at therapeutic concentrations. In dogs, M5 and tPA induced lysis of a femoral artery thrombus with comparable efficacies, but M5 caused ten-fold less (p=0.026) bleeding from wound sites due to an apparent sparing of hemostatic fibrin. This was postulated to be due to the finding that plasminogen activation by M5 was not promoted by intact (hemostatic) fibrin, and to the efficient inhibition of tcM5 by plasma C1-inactivator.

### Example 2

### Clot Lysis In Vitro

Materials

Recombinant Lys300→His mutant (M5) prouPA expressed in Escherichia coli (E. coli) was prepared as previously described [Liu, et al., (2002), supra] and obtained from Dr. Paolo Sarmientos at Primm (Milan, Italy). Recombinant prouPA expressed in E. coli was obtained from Landing Science and Technology Company, Nanjing, China. Human C1-inhibitor concentrate prepared from human plasma was kindly supplied by ZLB Behring GmbH (Marburg, Germany). Human Complement factor four (C4) was obtained from Calbiochem, Torry Pines, Calif. Chromogenic substrates for uPA (S-2444) and plasmin (S-2251) were obtained from DiaPharma (West Chester, Ohio, USA). The chromogenic substrate for C1 esterase (Spectrozyme C1E) was obtained from American Diagnostica, Stamford, Conn.

Methods

Fibrinogen was measured as thrombin clottable protein. Plasma was diluted with 2 vol 0.06 M sodium phosphate, pH 6.1. One volume of thrombin (100 NIH U/ml, ThromboMax from Sigma, St Louis, Mo., USA) was added, mixed, and incubated for 30 min at 37° C. The clot was wound onto a wooden stick to express the diluted serum, rinsed in 5 ml of the buffer, and deposited into a tube containing 1 ml of 5% NaOH. After boiling for 1 min, the clot was dissolved and the protein measured spectrophotometrically at 280 nm.

Zymography was performed by the method of Granelli-Piperno and Reich [J Exp Med. 148: 223-234 (1978)] as modified by Vassalli, et al., [J Exp Med. 159: 1653-1658 (1984)] with sodium dodecyl sulfate polyacrylamide gel electrophoresis (SDS-PAGE) according to Laemmli. After electrophoresis, the gel was washed with agitation for 2 h in 2.5% Triton X-100 in water, followed by 1 h in 0.1 M Tris-HCl (pH 8.0) and then placed on an underlay consisting of 0.8% agarose (Agarose low melting, Fisher Biotech, Kent City, Mich., USA), casein (2% w/v; Carnation non-fat dry milk, Nestle, Glendale, Calif., USA) and plasminogen: (20 μg/ml) in 0.1 M Tris HCl (pH 8.0) With incubation, the electrophoretic bands of plasminogen activator produce a clear zone in the white casein. Plasminogen activator inhibitor complexes become active in this system and also show up as lytic zones.

Zymograms of the Two-Chain Activators in Plasma

The single-chain forms of M5 and of uPA were converted to two-chain by incubation with 0.2 μM plasmin (American Diagnostica, Greenwich, Conn., USA) for 45 min at 37° C. in 0.05 M Tris-HCl, 0.1 M NaCl, 0.01% Tween-80, 10 mg/ml BSA as previously described [Pannell, et al., Blood. 69:22-26 (1987)]. These were added to human bank plasma (5 μg/ml) and incubated at 37° C. for 1 h with time point samples taken for zymography (FIG. 7).

Inhibitor Complexes in Plasma and with Purified C-Inhibitor

FIG. 7 shows zymograms of plasma in which equal amounts (5 μg/ml) of tcM5 (A) or tcuPA (B) were incubated (0-60 min). With tcM5 (A), there was a progressive loss over time of the uncomplexed enzyme associated with the appearance within 5 min and progressive increase of lysis bands at ˜150 kDa, corresponding to complexes with C1-inhibitor. The last lane shows the complex which was formed when purified C1-inhibitor was incubated for 1 h with tcM5 (5 μg/ml) migrating in the same position With tcuPA (B), the uncomplexed enzyme persists in the plasma for a longer time and the C1-inhibitor complexes appear more slowly and are much less apparent. The complex with purified C1-inhibitor (last lane) is too faint to be visible on the print. The lower MW inhibitor complex in plasma (˜115 kDa) is with antithrombin, a known plasma inhibitor of tcuPA [Murano, et al., Blood 55: 430-436 (1980)]. This has about the same intensity as that with C1-inhibitor with tcuPA (but not with tcM5).

On the zymograms, both the free tcM5 but especially the tcuPA, appear as higher and lower molecular forms, the latter more degraded form is an inevitable by-product of the plasmin activation of their parent single-chain forms. Complexes with PAI-1 in the plasma are not visible due to the negligible concentration of this inhibitor relative to that of the activators.

C1-Inhibitor Inhibition of tcM5 and tcuPA

The two-chain forms of M5 and of uPA were incubated (10 μg/ml) with purified C1-inhibitor (250 μg/ml) at 37° C. in 0.05 M Tris-HCl, 0.1 M NaCl, 0.01% Tween-80, 10 mg/ml BSA. Samples were taken at time points and assayed for remaining activity by chromogenic assay (S-2444) (FIG. 8). The data were plotted directly (FIG. 10) and a non-linear regression for first-order logarithmic decay was performed using GraphPad Prism (version 3.03 for Windows, GraphPad Software, San Diego, Calif., USA) in order to obtain the half-life (t_{1/2}) for inhibition. The pseudo-first order inhibition rate constant was calculated from k′=0.693/(t_{1/2}×[tcuPA]).

FIGS. 8A and 8B are zymograms of incubation mixtures of equal amounts of tcM5 or tcuPA incubated in buffer in the presence of purified C1-inhibitor (250 μg/ml), corresponding approximately to its physiological concentration. Similar to the findings in plasma, a relatively rapid and progressive loss of the tcM5 free enzyme was seen associated with the appearance of prominent inhibitor complexes (FIG. 8A). By contrast, with tcuPA the free enzyme persisted due to only a modest formation of inhibitor complexes taking place during the incubation, reflecting the differences in their inhibition rates (FIG. 8B).

Kinetics of Inhibition by C1-Inhibitor

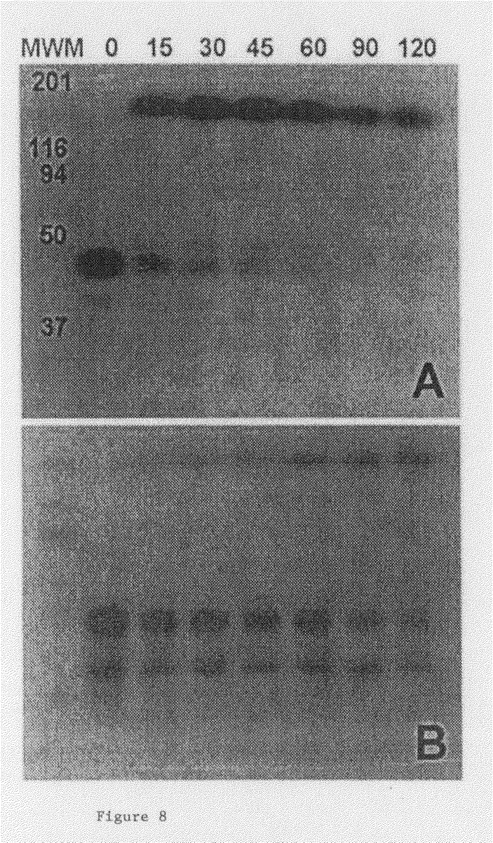

FIG. 9 shows the kinetics of inhibition of tcM5 compared with tcuPA in buffer containing C1-inhibitor (250 μg/ml) and 10 μg/ml (a high therapeutic concentration) of the two activators. At time points, synthetic substrate (S-2444) activity was measured and expressed as percent of uPA activity remaining. As shown, inhibition of uPA activity occurred at two very different rates. There was essentially complete inhibition of tcM5 within 60 min, whereas 30% of the tcuPA activity remained even after 3 h of incubation.

TABLE 1
Inhibition Kinetics.

| | t_{1/2} (min) | k′ (M⁻¹ sec⁻¹) |
|---|---|---|
| tcM5 | 10 | 5.78 × 10³ |
| tcuPA | 68 | 0.85 × 10³ |
| tPA ^{a} | | 0.008 × 10³ |

^{a} For melanoma single chain tPA from Murano, et al., Blood 55: 430-436 (1980); adjusted to normalize to the inhibitor concentration we used.

The inhibition rate of C1-inhibitor was about seven-fold greater against tcM5 than tcuPA. Huisman, et al., [Huisman, et al., (995), supra] previously reported that C1-inhibitor formed complexes with both single and two-chain tPA, but these complexes formed more slowly. C1-inhibitor has not previously been included among the plasma inhibitors of tcuPA [Murano, et al., (1980), supra]. As shown in Table 1, the inhibition of tcuPA was about 100-fold faster than that previously described for tPA by Huisman, et al., [Huisman, et al., (1995), supra] (recalculated to normalize to the inhibitor concentration we used).

Stability of M5 or prouPA in Plasma as a Function of Supplemental C1-Inhibitor

To determine the stability of the single chain forms of M5 and uPA in plasma, the M5 and prouPA were incubated (37° C. for 4 h) in plasma at a range of concentrations (10, 15, and 20 μg/ml) with and without extra C1-inhibitor added (250 μg/ml). Enzymatic activation of plasminogen was determined by measuring the plasminogen remaining (FIG. 10) by chromogenic assay (S-2251) after its activation with Streptokinase (2500 units/ml). C1-inhibitor also inhibits plasmin [Harpel. J Clin Invest. 49(3): 568-75 (1970)] which potentially complicates the assay. However, it was determined that SK:plasmin complexes, which are generated for this assay, are not inhibited by C1-inhibitor (unpublished observations).

Since prouPA is far less stable in plasma than M5, a separate study using far lower concentrations (2 and 4 μg/ml) incubated in plasma 2 h and 4 h±C1-inhibitor was also performed.

Promotion of M5 Stability in Plasma by C1-Inhibitor Supplementation

M5, like prouPA, is a single-chain zymogen which is activated by plasmin. Plasmin generation is triggered by the intrinsic activity of the proenzymes when they reach a certain threshold, such as at therapeutic concentrations. Conversion to the two-chain forms then amplifies plasminogen activation. Although the intrinsic activity of M5 is five-fold lower than that of prouPA [Liu, et al., (1996), supra], making its plasma stability or inertness that much greater, it nevertheless also has its concentration limits.

Therefore, the effect, of supplemental C1-inhibitor (250 μg/ml) added to plasma, on the concentration threshold at which instability occurs was evaluated. M5 or prouPA (10, 15, and 20 μg/ml) were incubated in plasma for 4 h with or without additional C1-inhibitor, after which the plasma plasminogen remaining was measured.

As shown in FIG. 10, at an M5 concentration of 10 μg/ml, non-specific plasminogen activation began to occur (˜25% loss of plasminogen) after 4 h, and at concentrations of 15 and 20 μg/ml, plasminogen activation by M5, as reflected by its depletion, was ˜85% and >90% respectively. However, with additional C1-inhibitor, plasminogen depletion was prevented at the 10 μg/ml M5 concentration, and at 15 and 20 μg/ml, reduced to ˜30% and ˜60% respectively. Therefore, C1-inhibitor supplementation was effective in promoting the plasma stability of M5 by raising the threshold concentration at which non-specific plasminogen activation occurred.

By contrast, as shown by the last two bars in FIG. 10, C1-inhibitor supplementation had no attenuating effect on plasminogen activation of prouPA at the high concentrations used. At much lower concentrations (2, 4, and 6 μg/ml) of prouPA, however, some attenuation of non-specific plasminogen activation by C1-inhibitor was seen (data not shown).

Fibrin-Specific Clot Lysis as a Function of Supplemental C1-Inhibitor

Clots were formed from 0.2 ml bank plasma by recalcification (35 mM) with the addition of a trace of thromboplastin and incubated at 37° C. for 1 h and overnight at room temperature. The following day, the clots were placed into 2.5 ml of bank plasma and M5 was added at 5 or 10 μg/ml. Lysis was determined by measuring the D-Dimer concentration in plasma samples removed at time intervals (FIG. 11A). The D-Dimer determinations were made independently by Dr. Gregory Gauvin, Mt Auburn Hospital, Cambridge, Mass. using a Beckman ACL 8000 analyzer. After lysis had gone to completion, aprotinin (500 KIU/ml) was added and the fibrinogen concentration determined and compared with that from a baseline sample (FIG. 11B).

The Effects of C1-Inhibitor on Clot Lysis and Fibrin Specificity

The attenuation of non-specific plasminogen activation by C1-inhibitor suggested that fibrin-dependent plasminogen activation and fibrinolysis might also be inhibited. This was anticipated from the clinical experience with PAI-1 in which higher levels have correlated clinically with fibrinolytic resistance, and also from our own laboratory findings in which a PAI-1 dose-dependent inhibition of clot lysis in a plasma milieu by prouPA was previously found (unpublished observations).

FIG. 11A shows representative clot lysis curves measured by the release of D-Dimer from a standardized plasma clot. Lysis induced by 5 or 10 μg/ml of M5 in plasma went to completion in 1½ and 2 hours, respectively, either with (open symbols) or without (closed symbols) C1-inhibitor supplementation (250 μg/ml). No detectable attenuation of the rate of M5-mediated fibrinolysis by the inhibitor was found (experiments done in triplicate), indicating that C1-inhibitor did not inhibit fibrin-dependent plasminogen activation.

By contrast, as seen in FIG. 11B showing the fibrinogen concentrations (as % of baseline) at the end of each clot lysis experiment, fibrinogenolysis was completely inhibited by supplementation with C1-inhibitor (indicated by the + symbol). Therefore, C1-inhibitor completely restored fibrin-specificity to the more rapid clot lysis rate otherwise achievable only at non-specific doses of the activator, i.e., at which fibrin degradation by excess plasmin is the rate-limiting factor.

Since plasmin is the common denominator of all plasminogen-activator mediated lysis, the findings that the fibrin-specific and non-specific rates (when plasmin is in excess), were equivalent are of special interest. They suggest that C1-inhibitor supplementation made a maximal fibrinolysis rate by M5 possible in vitro without sacrificing fibrin-specificity.

Evaluation of C1 Esterase Activity

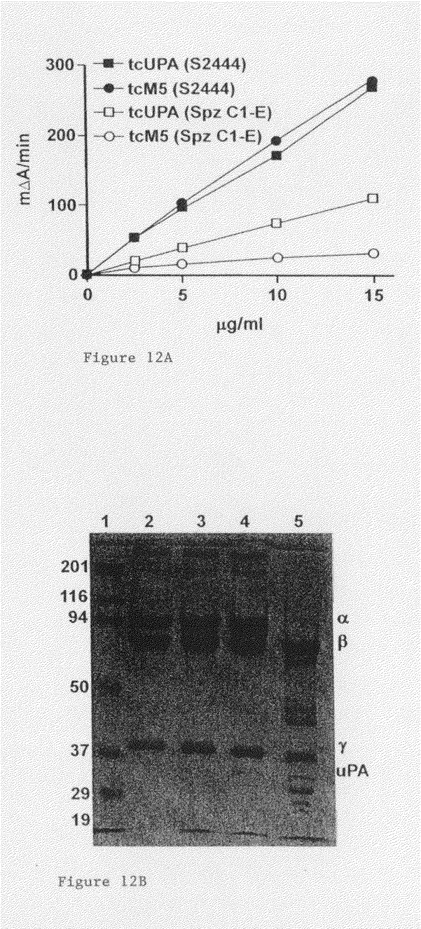

Since tcM5 was inhibited by the principal inhibitor of C1 esterase, the possibility that it may itself have some intrinsic C1 esterase activity was tested. Equal amounts (10 μg/ml) of tcM5 and tcuPA, adjusted to have equivalent activities against the uPA chromogenic substrate (S2444), were tested against the tripeptide chromogenic substrate Spectrozyme C1-E (480 μg/ml) (FIG. 12A).

Since Complement factor 4 (C4) is the natural substrate for C1 esterase, release of the ˜9 kDa peptide from the α-chain of C4 by the activators, using plasmin as a positive control, was also evaluated. A mixture of 10 μl of tcM5, tcuPA, plasmin (10 μg/ml) or buffer were incubated (37° C.) for 6 h with 40 μl C4 (480 μg/ml). Each of the incubation mixtures with C4, except plasmin, also contained aprotinin (100 KIU/ml). At the end of incubation, the mixtures were analyzed by SDS-PAGE under reducing conditions (FIG. 12B).

FIG. 12A shows that at concentrations of tcuPA and tcM5 which were equivalent against uPA chromogenic substrate (solid symbols), tcuPA had more activity against C1 esterase chromogenic substrate than did tcM5 (open symbols). A comparable difference in the same direction was found against the more general substrate, benzoyl-argininyl methyl ester (Sigma) (data not shown).

FIG. 12B shows an SDS PAGE under reducing conditions of the 6 h incubation mixtures of C4 with the following: buffer (lane 2), tcuPA (lane 3), tcM5 (lane 4) or plasmin (lane 5) (10 μg/ml). A faint band is discernable between the α and β chains of C4 in lanes 3 and 4, consistent with a shift of a trace amount of the α-chain by release of the peptide after a 6 h incubation with a high therapeutic concentration of either tcuPA or tcM5. By contrast, plasmin had a gross degradative effect, particularly of the α-chain.

The effect of plasmin is consistent with those reports in which complement activation and anaphylatoxin generation were found associated with therapeutic thrombolysis. This potentially deleterious side effect of plasmin further underscores the importance of limiting non-specific plasmin generation during fibrinolysis as much as possible.

C1-inhibitor is a major inhibitor of the complement pathway, specifically C1 esterase, but also has a number of other target serine proteases, including Factors XIIa, XIa, kallikrein, and tPA. The present findings indicate that tcuPA, and especially tcM5, need to be added to the list.

Due to the unusual interaction of tcM5 with C1-inhibitor, the question of whether tcM5 may itself have C1 esterase activity was raised. The activation of the complement system can cause damage to cells. When compared with an equivalent amount of tcuPA, tcM5 had, in fact, less activity against a C1 esterase synthetic substrate (FIG. 12A). Against its natural substrate, C4, both tcuPA and tcM5 at high therapeutic concentrations had a comparable effect consistent with release of a trace amount of anaphylatoxin after a six hour incubation. By contrast, plasmin induced a major degradation (FIG. 12B), consistent with reports of complement activation and anaphylatoxin generation during therapeutic thrombolysis [Bennett, et al., (1987), supra]. This data shows that M5 is safe and underscores the importance of limiting non-specific plasmin generation, as has been demonstrated with supplemental C1-inhibitor with M5.

A Method for Optimizing Thrombolysis without Inducing Non-Specific Plasminogen Activation and Bleeding.

The efficacy of therapeutic thrombolysis is limited by the non-specific plasminogen activation which induces a severe bleeding diathesis at the higher doses of all plasminogen activators. For example, dose-response trials with tPA demonstrated superior coronary thrombolysis with 150 mg per patient. However, at this dose, the hemorrhagic complications were unacceptably high [Califf, et al., Hemorrhagic complications associated with the use of intravenous tissue plasminogen activator in the treatment of acute myocardial infarction American J of Med, 85:353-59], obliging the adoption of 100 mg per patient. Bleeding in this study was significantly associated with nadir fibrinogen levels, a marker of non-specific plasmin generation.

M5, a single site mutant of proUK, is the exception to this plasminogen activator rule which has heretofore placed a strict upper limit on dosage and thrombolytic efficacy. This exception is related to the unpredicted and unexpected fact that the enzymatic, two-chain form of M5 (tcM5) is inhibited by C1-inhibitor (C1INH) in plasma at a rate sufficient to prevent all non-specific plasminogen activation but insufficient to inhibit fibrin-dependent plasminogen activation, i.e., fibrinolysis.

Therefore, by supplementing the endogenous concentration of C1INH, it is possible to achieve maximum fibrinolysis without inducing any non-specific effects, as evidenced by fibrinogen degradation. In order to achieve this, it is necessary to increase the average C1INH concentration (250 mcg/ml) 3-4 fold.

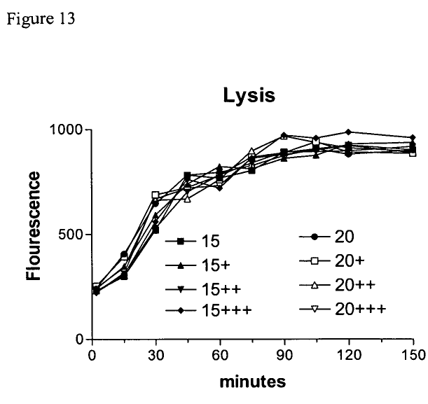

The effect of C1INH on fibrinolysis and fibrinogenolysis is illustrated in FIGS. 13-15 of experiments in which standard clots, made from clotted human plasma, were suspended in plasma to which a range of high doses of M5 and 500-750 μg of C1INH were added.

In FIG. 13, curves of clot lysis experiments in a plasma milieu at high doses of M5 are shown. As shown, the clot lysis curves were very similar at these doses. This was because they are at the maximal rate achievable.

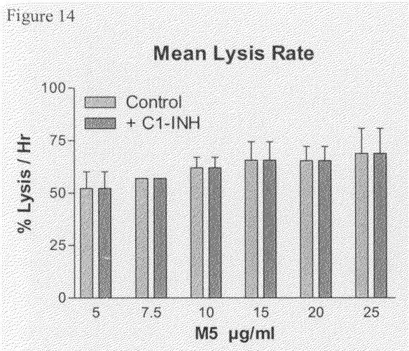

In FIG. 14, bar graphs show the mean lysis rate and standard deviation (SD) of 3 experiments each at the range of doses (5-25 μg/ml) of M5 used. The mean lysis rate was calculated from the time point at which maximal fluorescence released from the fluorescence-tagged fibrin divided by the time. A dose response of up to 15 μg/ml was found after which there was a leveling off of the mean lysis rate indicating that a maximum had been reached.

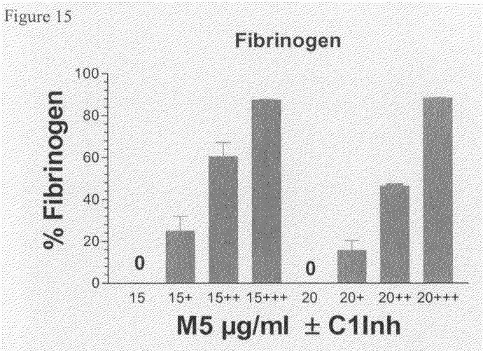

In FIG. 15, a bar graph showing the percent (%) fibrinogen remaining in each of the plasma samples corresponding to the clot lysis experiments shown in FIG. 13. The mean values and SD are shown. At each of the two doses no additional C1INH or 250, 500 or 750 μg of C1INH indicated by +, ++ or +++ was added. As shown, without added C1INH all of the fibrinogen was degraded (indicated by the 0), meaning that extensive non-specific plasminogen activation with plasminemia causing fibrinogen degradation, had taken place. When this occurs in vivo there is a generalized hemorrhagic state that is induced associated with extensive bleeding. This undesirable side effect of rapid fibrinolysis is progressively inhibited by C1INH. This is an effect that is unique to M5 and means that with this activator, optimal (i.e., maximal) lysis rates are possible when C1INH is administered up to, for example, a plasma concentration of 1,000 μg/ml.

What is claimed is:

1. A method of increasing the efficacy and reducing bleeding complications of fibrinolysis treatment of a patient, the method comprising:
a) providing a pro-urokinase mutant polypeptide (M5) wherein the amino acid Lysine at position 300 has been replaced by the amino acid Histidine;
b) administering an amount of the M5 sufficient to cause efficient lysis of an occlusive blood clot; and
c) administering an amount of exogenous C1-inhibitor sufficient to limit non-specific plasminogen activation in blood and its attendant bleeding complications.

2. The method of claim 1 wherein the C1-inhibitor is administered in an amount sufficient to establish a concentration, in the plasma of the patient, that is within the range of about 1.5 to about 4 times the mean physiological level.

3. The method of claim 1 wherein M5 is mixed with a pharmaceutically acceptable carrier and administered as a bolus of about 20 to about 60 mg.

4. The method of claim 1 wherein M5 is mixed with a pharmaceutically acceptable carrier and administered by intravenous infusion at a rate of about 80 to about 300 mg/hour.

5. A method of inhibiting the non-specific plasminogen activation by the two-chain urokinase plasminogen activator mutant, tcM5, comprising administering, to a patient being treated with M5, an amount of exogenous C1-inhibitor sufficient to inactivate free tcM5 generated from M5 activation, wherein M5 is a pro-urokinase plasminogen activator mutant polypeptide in which the amino acid Lysine has been replaced by Histidine at position 300.

6. The method of claim 5 wherein the C1-inhibitor is administered in an amount sufficient to establish a concentration, in the plasma of the patient, that is within the range of 1.5 to 4 times average normal physiological levels.

7. A method of preventing side effects during high infusion-rate fibrinolysis in a patient, the method comprising:
a) providing a pro-urokinase mutant polypeptide (M5) wherein the amino acid Lysine has been replaced by the amino acid Histidine at position 300;
b) administering an amount of the M5 sufficient to cause a maximal rate of fibrinolysis by a plasminogen activator, wherein the maximal rate is defined as the rate at which the plasmin degradation of fibrin, rather than the plasminogen activation by M5, is the rate limiting step in clot lysis; and
c) administering an amount of exogenous C1-inhibitor sufficient to limit non-specific plasminogen activation by M5, whereby the C1-inhibitor inhibits non-specific plasminogen activation resulting in degradation of clotting factors I, V, and VIII and bleeding.

8. A method of increasing the plasma stability or inertness of M5 during fibrinolysis comprising administering an amount of exogenous C1-inhibitor sufficient to establish a concentration of C1-inhibitor that is within the range of 1.5 to 4 times the mean physiological level, wherein the C1 inhibitor inhibits non-specific plasminogen activation by tcM5 without inhibiting clot lysis or fibrin-dependent plasminogen activation by M5.

9. A method of accelerating the rate of fibrinolysis while at the same time preventing fibrinogen degradation in a patient comprising:
a) providing M5;
b) administering an amount of M5 sufficient to establish M5 concentrations of up to 10-15 μg/ml in the plasma of the patient; and
c) administering an amount of exogenous C1-inhibitor sufficient to prevent fibrinogenolysis at these M5 concentrations, meaning establishing a concentration of C1-inhibitor, in the plasma of the patient, that is within the range of 1.5 to 4 times the mean physiological level;
wherein non-specific plasminogen activation by M5 is prevented.

10. A method of increasing the dose-range of fibrin-specific clot lysis by M5 in a patient, comprising administering an amount of exogenous C1-inhibitor sufficient to prevent non-specific plasminogen activation by M5.

11. The method of any of claims 1, 5, 7, 8, 9 or 10, wherein the C1-inhibitor is selected from one or more from a group consisting of native C1-inhibitor from plasma, recombinant C1-inhibitor from mammalian cells, E coli or yeast, serpin domain deletion mutants of recombinant C1-inhibitor, transgenic C1-inhibitor, chimeric C1-inhibitor, non-glycosylated C1-inhibitor and differently glycosylated C1-inhibitor.

12. The method of any of claims 1, 5, 7, 8, 9 or 10, wherein said C1-inhibitor administration is selected from one or more of a group consisting of being administered prior to or simultaneously with M5.
